(19) United States
(12) Patent Application Publication
(10) Pub. No.: US 2024/0366073 A1
(43) Pub. Date: Nov. 7, 2024

(54) MEDICAL IMAGING DEVICE, IN PARTICULAR AN ENDOSCOPE OR EXOSCOPE

(71) Applicant: KARL STORZ SE & Co. KG, Tuttlingen (DE)

(72) Inventors: Hannes KÖHLER, Tuttlingen (DE); Annekatrin PFAHL, Tuttlingen (DE)

(21) Appl. No.: 18/293,207

(22) PCT Filed: Aug. 10, 2022
(86) PCT No.: PCT/EP2022/072450

§ 371 Date: Jan. 29, 2024

(30) Foreign Application Priority Data
Aug. 12, 2021 (DE) 10 2021 121 025.9

(51) Int. Cl. A61B 1/00

(52) U.S. Cl. CPC A61B 1/00193; A61B 1/00186

(57) ABSTRACT

The invention relates to a medical imaging device, in particular a stereo endoscope or stereo exoscope, comprising: a light source having a light spectrum; an optical system, which has a first optical path, a first image sensor having a first sensor filter, a second optical path, and a second image sensor having a second sensor filter; wherein the relevant optical path extends between a viewing region and the relevant image sensor such that the first image sensor records a first image of the viewing region by means of the first optical path, and the second image sensor records a second image of the viewing region by means of the second optical path; wherein a first filter is or can be introduced into the first optical path by means of a first filter positioning device and/or a second filter is or can be introduced into the second optical path by means of a second filter positioning device, such that, from filtered image information of the first image and/or filtered image information of the second image, different spectral ranges of the relevant image can be evaluated, on the basis of the relevant filter introduced into the relevant optical path, in order to obtain additional image information or several pieces of additional image information relating to the physiological parameters in the viewing region.

The invention relates to a medical imaging apparatus, in particular an endoscope or an exoscope, having a light source with a light spectrum, an optical unit with at least a first optical path and a first image sensor with a first sensor filter, and at least a second optical path with a second image sensor with a second sensor filter, wherein the respective optical path extends between an observation region and the respective image sensor such that the first image sensor records a first image of the observation region by means of the first optical path and the second image sensor records a second image of the observation region by means of the second optical path, wherein the light source is configured to illuminate the observation region with a light spectrum and the first optical path is assigned a first filter with a first filter spectrum and/or the second optical path is assigned a second filter with a second filter spectrum such that, especially in real time, physiological parameters of the observation region are determinable depending on the light spectrum and a further piece of image information of the observation region by means of a respective filtered piece of image information from the first image and/or the second image.

In this context, for example endoscopes with two image recording paths, in particular so-called stereo endoscopes or stereo exoscopes, which can record or display a spatial image of an observation region on account of for example a stereoscopic observation with a first optical path and a second optical path, are known as medical imaging apparatuses.

Furthermore, endoscopes or else endoscope systems which are able to record and display physiological parameters of an observation region by means of a corresponding illumination with specific light spectra and a corresponding evaluation by means of one image sensor or more image sensors are known. For example, so-called multispectral endoscopes are thus known, by means of which a conclusion can be drawn, for example, with regard to an oxygen saturation or else a fat content, a hemoglobin content or any other parameter within the observation region by way of illuminating the observation region using defined light spectra and correspondingly evaluating the correspondingly radiated-back light spectra. For example, an oxygen content of the observed tissue thus can be directly determined and/or monitored during a surgical procedure by the use of such a multispectral imaging endoscope.

In this context, known medical imaging apparatuses are disadvantageous in that physiological parameters cannot be determined in parallel with for example a display of a real image of the observation region or, if these actions are performed in parallel, a limitation in terms of the temporal, spatial and/or spectral resolution must be tolerated.

It is an object of the invention to improve the prior art.

The object is achieved by a medical imaging apparatus, in particular an endoscope or an exoscope, having a light source with a light spectrum, an optical unit with at least a first optical path and a first image sensor with a first sensor filter, and at least a second optical path with a second image sensor with a second sensor filter, wherein the respective optical path extends between an observation region and the respective image sensor such that the first image sensor records a first image of the observation region by means of the first optical path and the second image sensor records a second image of the observation region by means of the second optical path, wherein the light source is configured to illuminate the observation region with a light spectrum and the first optical path is assigned a first filter with a first filter spectrum and/or the second optical path is assigned a second filter with a second filter spectrum such that, especially in real time, physiological parameters of the observation region are determinable depending on the light spectrum by means of a respective filtered piece of image information from the first image and/or the second image, wherein the first filter is introducible into the first optical path by means of a first filter positioning device and/or the second filter is introducible into the second optical path by means of a second filter positioning device, such that a filtered piece of image information from the first image and/or a filtered piece of image information from the second image, depending on the respective filter introduced into the respective optical path, render/renders evaluable different spectral regions of the respective image in order to obtain one piece of additional image information or more pieces of additional image information regarding the physiological parameters in the observation region. The configuration according to the invention of a medical imaging apparatus in particular allows appropriate pieces of information to be obtained in parallel, in particular next to one another in real time. In the process, the appropriate pieces of information are for example created and provided in such a way that a user experiences a simultaneous display of the appropriate pieces of information.

In this case, “real time” describes an implementation of technical or else electronic procedures such that a reliable processing, display, and/or representation of the procedures is implemented within a defined period of time. In the narrower sense, the term “real time” is also used in the sense that, for example, an operator gains the impression of simultaneity of events, which is to say for example the perception of a “real time” representation in accordance with the real impression of time for the operator. For example, there is thus a representation in parallel with a frame rate above 24 frames per second, or else a higher frame rate, with the result that an operator can no longer make a distinction between individual frames.

By way of a few components and a comparatively simple structure, a medical imaging apparatus designed thus combines for example an endoscope or an exoscope, a dual image endoscope or else a stereoscopic endoscope or a stereoscopic exoscope or any other dual or stereoscopic medical imaging apparatus with the capability of evaluating the observation region with regards to physiological parameters on the basis of the respectively chosen filter introduced into the respective optical path. In particular, depending on the design of the respective filter, a spectral evaluation can then be performed here from a piece of difference information between the first optical path and the second optical path or by means of a combined piece of information from the first optical path and the second optical path, with the result that, next to a dual image or else in addition to a dual image, the determination of physiological parameters of the observation region is made possible by means of the available imaging technology.

Thus, for example, a piece of difference information can also be formed from the first optical path and the second optical path by means of the first image sensor and by means of the second image sensor, with the result that spectral components accordingly filtered out by a respectively other filter in the respectively other optical path can for example be read from the then respectively other optical path and used.

The following terms are explained in this context:

A “medical imaging apparatus” can be any technical and/or electronic device suitable for recording, further processing, and/or transmitting an image of an observation region in medical surroundings, and for example for displaying said image on an electronic visual display. By way of example, such a medical imaging apparatus is an endoscope, a dual endoscope, a stereo endoscope, an exoscope, or a stereo exoscope. In this case, such an “endoscope” is an imaging apparatus with usually a narrow and elongate design, which is suitable for inserting said apparatus into a cavity or through a usually small opening and for recording, in the case of a “stereo endoscope” by means of two cameras or two image sensors, an image of an observation region within the cavity and/or the region located behind the small opening. An “exoscope” is a comparable device, which is used for example from the outside for imaging purposes during medical procedures, which is to say within the scope of what is known as an open surgical procedure. In this case, the “stereo” property of the respective endoscope or exoscope describes the capability of recording a stereoscopic image of the observation region by means of two optical paths and/or two optical units. A corresponding dual endoscope or dual exoscope is able to record two separate images, without for example a stereoscopic reconstruction being implemented. Attention is drawn in this context to the fact that a respective “endoscope” in the strict sense, as described above, may also be linked within an endoscope system to further devices, for example a cable guide, further sensors, and/or display equipment for displaying a piece of image information on an external monitor. Further, there frequently is no strict separation between the use of “endoscope” and “endoscope systems”, and these terms are sometimes used synonymously.

A “light source” is for example an LED, an incandescent lamp, or any other light-emitting device. Further, such a light source may also be realized by virtue of a light created by means of an LED or any other light-creating device being steered or directed to a corresponding location at the observation region by means of, for example, a light guide, which is to say for example an optical fiber or an optical fiber bundle. In this case, such a light source serves to illuminate the observation region with light with appropriate light spectra.

In this case, a “light spectrum” describes the wavelength range and/or an intensity distribution over various wavelengths, in which the respective light source emits light. For example, such a light spectrum can in this case be depicted graphically in the form of a diagram of the illumination intensity against a respective wavelength.

An “optical unit” denotes the totality of all components which steer light and/or a piece of image information or an image along the optical path. By way of example, such an optical unit comprises lenses, covers, protective screens, or else filters here.

An “optical path” is in particular the path traversed by light of a corresponding image from the observation region via a respective optical unit to for example a respective image sensor. For example, such an optical path is defined here by an optical axis or as a geometric profile.

By way of example, an “image sensor” is an electronic chip or any other equivalent device, by means of which light running along the optical path and through the respective optical unit and/or a corresponding image can be recorded and converted into electronic signals. By way of example, such an image sensor is a CCD chip or a comparable electronic component.

A “sensor filter” describes a filter usually assigned to a respective image sensor or a corresponding filter device, which is suitable for advance filtering of the light that is incident on the image sensor for recording by the image sensor. By way of example, the image sensor comprises a sensor filter which supplies parts of the image sensor assigned to corresponding color values with light that has been respectively prefiltered in accordance with the color. Thus, a typical image sensor may for example have an RGB filter in front of corresponding sensor parts for individual pixels for example, with the result that predominantly only the respective piece of information in relation to R (red), G (green), and B (blue) is fed to a respective pixel. In this case, every so-called pixel of the image sensor usually has at least three component pixels, which are fed the piece of R, G, B-information by way of an appropriate filter, with the result that a differentiated color display is made possible thereby from the pixel formed from the component pixels. Inter alia, image sensors with what is known as a Bayer filter are also known in this context.

An “observation region” describes the region, the volume or the area which is intended to be observed by means of the medical imaging apparatus and of which a corresponding image is intended to be taken. By way of example, in this case such an observation region is an organ, a bone, a portion of a human or animal body, or any further region of interest for a corresponding observation.

A “filter” is an optical component and hence can be part of the designated optical unit. In particular, the action of a filter is such that certain wavelength ranges of a complete light spectrum are damped or reduced or transmitted, or else completely hindered from traveling further along the optical path. In this context, such a filter has a corresponding “filter spectrum”, which, in a manner analogous to a light spectrum, describes the respective intensity of transmitted or retained wavelengths of the filter. Here, reference is made both to a transmission filter spectrum, which describes the transmitted intensity component in the respective light spectrum, and a degree of retention, which specifies what component is not transmitted by the filter. For example, so-called graduated filters are known in this context; their filtering effect changes continuously over a filter surface. Furthermore, so-called edge filters are known, which respectively retain or pass separated spectral regions quite selectively.

In this case, a “piece of filtered image information” is a piece of image information which has passed through the respective filter, and hence the wavelength components defined according to the respective filter spectrum have been scrubbed or reduced therefrom.

In the present case, “physiological parameters” of the observation region are for example oxygen concentrations, fat contents, perfusion values, hemoglobin concentrations, or else a water content, for example in a considered organ and/or in the tissue of the respective organ in the observation region. By way of example, such physiological parameters are ascertainable by means of appropriate light spectra by virtue of an absorptance for a wavelength or an appropriate wavelength range or else a plurality of absorptances for a wavelength or for a plurality of wavelength ranges of a light spectrum being analyzed and this being used to draw conclusions about an appropriate physiological parameter. For example, a specific absorption wavelength or a plurality of absorption wavelengths or else certain absorption wavelength ranges are thus assigned to a hemoglobin concentration, another absorption wavelength or a plurality of such absorption wavelengths or else absorption wavelength ranges are assigned to a water content, or a third absorption wavelength or a plurality of absorption wavelengths or absorption wavelength ranges are assigned to an oxygen content in the blood. In this case, appropriate wavelength ranges for establishing different physiological parameters can be the same, overlapping or different, or can be used in different combinations.

A “filter positioning device” is a technical device suitable for moving the first filter, the second filter or a respective filter between a position located outside of the optical path and a position located within the optical path. For example, such a filter positioning device is an apparatus, an arrangement with a linear guide or any other technical device, wherein the filter positioning device can position the respective filter in the respective optical path such that the effect of the respective filter is developed in the optical path, which is to say light propagating along the optical path is filtered accordingly. In that case, this effect is not developed outside of the optical path because for example the respective filter is not physically arranged in a propagation path of the light.

To provide further variants of evaluating pieces of additional image information regarding the physiological parameters, the first optical path and/or the second optical path is or are assigned a third filter, a fourth filter, and/or a further filter, wherein the third filter is introducible by means of a third filter positioning device, the fourth filter is introducible by means of a fourth filter positioning device, and/or the further filter is introducible by means of a further filter positioning device into the first optical path and/or into the second optical path.

In an embodiment, the first filter, the second filter, the third filter, the fourth filter, and/or the further filter in this case have different filter spectra from one another.

In this case, filter spectra that are “different from one another” are those filter spectra in particular, by means of which different spectral ranges of the light passing through the respective filter are filtered out or blocked. For example, the first filter has properties which filter out light at a wavelength of 680 nm whereas the second filter has properties which filter out light at the wavelength of 730 nm. It is self-evident that appropriately other wavelengths or else wavelength ranges may also satisfy this example.

To be able to jointly use certain filter states with filters matched to one another for the purpose of establishing specific physiological parameters, the first filter, the second filter, the third filter, the fourth filter, and/or the further filter are grouped, in particular in a group of two filters assigned to one another, such that filters in each case assigned to one another in the group are jointly introducible into the first optical path and into the second optical path.

In this case a “group” of filters is a set of in each case cooperating filters in the first optical path and in the second optical path, which filters are suitable for establishing appropriately desired physiological parameters by means of a difference in the filter spectra or filter spectra that differ from one another. In that case, such filters of a group are “jointly introducible” in such a way that respective filters assigned to one another in the group are jointly introduced into the first optical path and the second optical path or, in an alternative state, are each arranged outside of the respective optical path.

In an embodiment, the first filter, the second filter, the third filter, the fourth filter, and/or the further filter are grouped with one another in an operational group, in particular in a respective operational group made of two filters assigned to one another, such that a first mode of operation, a second mode of operation, a third mode of operation, and/or a further mode of operation is selectable by a user by means of an input, wherein a selection can be made from a group of respective filters for different modes of operation, for example a group of the first filter and the second filter for an oxygen saturation mode of operation, a group of the third filter and the fourth filter for a water content mode of operation, and/or a group of two further filters for a hemoglobin content mode of operation.

An “operational group” can be a group of filters assigned to a specific “mode of operation”. In this case, such a “mode of operation” describes a mode which is selectable by for example an operator and which can be preselected for example using a switch of a medical imaging apparatus and hence can be selected, with the result that filters belonging appropriately to the mode of operation or filter pairs of a group belonging to the mode of operation are then introduced into the respective optical paths.

To be able to meaningfully use an appropriate group or else an appropriate operational group, the filter positioning device associated with a respective mutually assigned filter is assigned to a filter positioning system and operatively connected to the latter such that the respective group or operational group of in particular two filters assigned to one another is jointly introducible into the first optical path and into the second optical path by means of the filter positioning system.

For example, a “filter positioning system” describes a mechanical set of various positioning devices such that only a joint introduction of mutually assigned filters in the respective optical path is rendered possible purely by the mechanical coupling. For example, such a filter positioning system is a joint frame for two or more different filters or else a system in which appropriate filter positioning devices are coordinated and synchronized by means of an electronic or electrical coupling.

In this case, continuous imaging can also be implemented in each case in such a way that a group and/or an operational group is selected and there is an evaluation with regards to the physiological parameters together with the recording and/or in parallel with the recording of the image information.

In particular, “continuous imaging” describes here the process of implementing no switchover to other filters, other groups or other operational groups but instead implementing a continuous readout of the pieces of image information from a fixed group or a fixed operational group, and hence both physiological parameters and a piece of image information, for example an RGB image in the visible region, are recorded virtually “in parallel”.

In a further embodiment, the respective filter or the respective filters is or are introducible into the respective optical path by means of being pushed in, by means of being pivoted in, and/or by means of being screwed in.

In this case, “pushing in” denotes a linear or substantially linear guidance of the respective filter or a respective group or operational group of filters, wherein this pushing in is implemented, in particular, at an angle of for example 90° with respect to the respective optical path in the process. Here, the angle of 90° is mentioned by way of example; pushing in can likewise be implemented in accordance with an angle ≠0° with respect to the optical path. Angle details refer here to a full angle of 360°.

“Pivoting in” describes a rotational movement of a respective filter or of a group or else operational group of respective filters, wherein this rotation is implemented about a reference axis at right angles to, or at an angle ≠0° with respect to, the optical axis of the filter.

By contrast, “screwing in” is implemented as a rotational movement about an axis arranged parallel or substantially parallel to the optical axis of the respective filter or to optical axes of the group or operational group of different filters.

However, an introduction of the respective filter or the respective filters by means of a coupled movement made up of pushing in, pivoting in, and/or screwing in is likewise possible.

To realize the function of the medical imaging apparatus easily and using available means, the respective filter positioning device or the respective filter positioning system comprises or comprise a drive such that the respective filter or the respective filters assigned to one another is or are introducible into the first optical path and/or into the second optical path by means of the respective drive, wherein the drive in particular comprises a stepper motor, a gearmotor, a lifting magnet, a pivoting magnet, and/or a manually operable drive.

In the present example, a “drive” describes a technical device used to move, displace or influence, in a differently realized manner, the filter positioning device or the filter positioning system. Such a drive is frequently also referred to synonymously as a motor and for example also comprises a gearing of different forms for converting a force or a torque.

A “stepper motor” is for example a synchronous motor in which the rotor can be rotated through a small angle, specifically a so-called “step” or a corresponding multiple of such a step by a controlled, for example incrementally rotating, electromagnetic field of corresponding stator coils. Corresponding stepper motors are known both as rotational motors and as linear motors. For example, such a stepper motor is a reluctance stepper motor, a permanent magnet stepper motor, a hybrid stepper motor, or else a Lavet-type stepper motor.

A “gearmotor” is a motor, for example an electric motor or else a magnetic motor, which has an additional gearing for transmission of a rotational speed and/or a torque.

A “lifting magnet” is a magnetic drive in which a linear movement is triggered by means of magnetic forces, which is to say under electromagnetic excitation. By contrast, a “pivoting magnet” is a drive in which a rotation is triggered by means of magnetic forces.

In the simplest case, a “manually operable drive” describes a slider, a switch, or a lever mechanism, by means of which appropriate filters or appropriate filter groups can be introduced into the respective optical paths or can be removed therefrom again. In the process, such a manually operable drive may also comprise appropriate gearings, transmissions or other additional devices, for example a lever transmission or a gear transmission.

In an embodiment, the first filter, the second filter, the third filter, the fourth filter, and/or the further filter is an edge filter or a band filter.

An “edge filter” has two sharply separated spectral regions in which the edge filter transmits, which is to say is transmissive, or absorbs, which is to say is non-transmissive. In this case, such edge filters are implemented as what are known as high-pass filters, which is to say with a transmission above a certain wavelength, or as low-pass filters, which is to say as edge filters with a transmission below a certain wavelength.

A “band filter” is a corresponding filter which comprises a plurality of edge filters, which is to say for example transmits or absorbs what is known as a “band” of spectral components between a lower wavelength and an upper wavelength. Depending on whether such a filter transmits or blocks a wavelength range, reference is made to a “bandpass filter” or “bandstop filter”. Hence, a separation is implemented with a respective edge filter at each of the lower wavelength and the upper wavelength.

To be able to effectively also use the medical imaging apparatus for imaging in the visual range in addition to the determination of physiological parameters and for example to also be able to use spectral components of an optically uniformly visible piece of image information, the first filter, the second filter, the third filter, the fourth filter, and/or the further filter have a respective filter spectrum such that by means of the different filter spectra of the respective filters by means of two filters assigned respectively to one another in the first optical path and in the second optical path it is possible, by way of a parallel evaluation of the first image and the second image, to determine a color image of the observation region and to determine one piece of additional image information or more pieces of additional image information regarding the physiological parameters together.

In this case, a “parallel evaluation” describes an evaluation of corresponding images as closely together in time as possible, wherein for example in contrast to a switchover between different manners of observation in different so-called “frames”, which is to say images recorded directly in succession, required in the prior art there is an evaluation such that an observer no longer perceives a difference between these frames, which is to say the impression of a simultaneous evaluation arises for the observer. For example, a so-called “frame rate”, which is to say a repetition rate of appropriately recorded images, is so high that the repetition frequency is above a frequency perceivable by the observer.

For example, a “color image” is an image visible to the observer in true colors or an image rendered visible on a monitor for example, which image maps a color-based reality in the observation region as accurately as possible.

In a further embodiment, the light source is a white-light source, and so the light source illuminates the observation region with white light.

Reliable recording of the color image is possible by means of this illumination with white light, for example implemented additionally or at the same time.

“White light”, which is also referred to as “polychromatic light”, describes light which consists of a mixture of different colors, which is to say a mixture of different spectral components. Such light is therefore also described as being spectrally broadband. In this case, such white light may for example also be white light within the meaning of daylight or apparently white light from a typical “white” illumination source, but also any other mixture of light wavelengths. By way of example, such white light may also accordingly include, in superimposed form, wavelengths suitable for determining physiological parameters.

In order to be able to realize the medical imaging apparatus in particularly compact fashion and with a few components, the optical unit comprises a beam splitter, wherein the beam splitter is used to split incident light for an image of the observation region into light components for the first image and light components for the second image such that the first image is suppliable to the first image sensor via the first optical path and the second image is suppliable to the second image sensor via the second optical path, wherein the beam splitter in particular comprises a prism, a semi-transparent mirror or two semi-transparent mirrors, and/or a mirror.

A “beam splitter” is an optical component which separates an individual light beam or a bundle of individual light beams into two component beams or two component bundles. As a result of this property, a beam splitter can be used to split a light beam among for example two image sensors. For example, such a beam splitter is realized as a prism or by means of semi-transparent or complete mirrors.

In optics, a “prism” describes a component in the form of a geometric prism which, as a result of its geometry and corresponding refractive properties of its material, brings about different optical effects. For example, light can be deflected or else separated into different spectral ranges by means of a prism. Prisms are also known for splitting light into different polarization directions.

A “mirror” is an optical device which comprises a surface that completely or partly reflects incident light. A “semi-transparent mirror” in this context is a mirror in which first components of a light beam are transmitted and further components of the light beam are reflected. Such so-called semi-transparent mirrors are known with different degrees of reflection, which is to say with a different ratio of transmitted to reflected light. Such a semi-transparent mirror, which for example is installed in an optical path at an angle of 45°, can be used to deflect reflected components out of the optical path and for example steer said reflected components to a separate image sensor and guide transmitted component parts further along the optical path to an image sensor located in the optical path.

In a further embodiment, the first optical path and the second optical path are arranged spatially offset from one another such that the first image sensor records a first image of the observation region by means of the first optical path and the second image sensor records a second image of the observation region by means of the second optical path.

Such an arrangement can be used to record what is known as a dual image of the observation region, in which different observation angles and/or different observation axes are in each case used in the first image and in the second image for the purpose of creating the respective image.

In this case, “spatially offset from one another” describes two optical paths arranged next to one another in the simplest case, with the result that a respective image can be recorded from two perspectives. Further, a parallel or skew arrangement of corresponding optical axes along the optical paths can in particular also be configured and used, with the result that it is possible to record what is known as a stereoscopic image. This is made usable in an embodiment of the invention described hereinafter. In this case, such a stereoscopic image imitates the spatial view of a living being, by virtue of a respective image being recorded from at least two perspectives. Subsequently, a spatial impression can be reconstructed or created from the various pieces of information of the respective images from different perspectives. This is also described as “stereoscopically forming a piece of spatial image information”.

To be able to best possibly exploit this chosen spatially offset arrangement, the first image and the second image are assigned to one another in an overlaid image to form a piece of dual image information, wherein in particular the formation of the piece of dual image information in the overlaid image comprises a reconstruction of a correlation between the first image and the second image on the basis of a piece of respective filtered image information, in particular a reconstruction of a disparity, wherein the reconstruction is implemented in particular on the basis of pieces of image information with wavelength spectra transmitted by the respective filter, in particular as a result of illuminating the observation region with white light.

In this case, a “piece of dual image information”, also referred to as a dual image, is a piece of image information which is put together from two pieces of image information and which for example comprises an image representation of two images, recorded by means of different optical paths, of the observation region, without these images needing to be stereoscopic.

In this case, a “reconstruction of a correlation” describes associating, between the first image and the second image, an appropriate picture element belonging to a picture element in the observation region or a corresponding piece of partial information, whereby for example an offset of the first image from the second image is calculated and used as a correction for the formation of the piece of dual image information.

In an embodiment, the formation of the piece of dual image information comprises a stereoscopic formation of a piece of spatial image information for the observation region.

Such a piece of spatial image information thus allows an observer to for example record spatial conditions in the observation region and use these to operate the medical imaging apparatus or to assist an action with the medical imaging apparatus.

In this case, a “piece of spatial image information” is the piece of information which allows a conclusion to be drawn for example with regards to a topography of the observation region and/or a spatial arrangement of corresponding objects within the observation region. In this case, a piece of spatial image information can for example be an image displayed as a 3-D image, which provides an observer with a detailed piece of information about the topography of the observation region. Furthermore, such a piece of spatial information may also arise from the fact that, for example, the respective piece of image information for a respective eye is supplied to an observer, with the result that the actual “spatial formation” is carried out by the observer since spatial vision is physiologically suggested to the observer. By way of example, this can be implemented by means of VR (virtual reality) goggles, which are then supplied with a “piece of dual image information” within the meaning of the description above.

In order to be able to obtain precise pieces of information about the physiological parameters, the medical imaging apparatus comprises an evaluation unit which is configured to evaluate the first image and/or the second image in relation to a hemoglobin index, water index, oxygen index, and/or an oxygen-infrared index, with the result that a hemoglobin content, a water content, an oxygen concentration, and/or else a presence of an adjuvant, in particular a fluorescent substance, is determinable.

For example, such an “evaluation unit” comprises a memory in this case, in which a method for evaluating the medical imaging apparatus is stored. Furthermore, the evaluation unit may comprise a processor or microcontroller, wherein the evaluation method is executable using the processor or the microcontroller.

In a further embodiment, the medical imaging apparatus comprises a display unit which is configured for the simultaneous, superimposed and/or correlated display of the piece of first image information, the piece of second image information, the piece of dual image information, the stereoscopically formed piece of spatial image information and/or the pieces of additional image information.

In this case, such a “display unit” is for example a PC, a minicomputer or an electronic visual display with a corresponding processor, which is able to display both the piece of first image information, the piece of second image information, a piece of spatial image information formed therefrom, and/or also a piece of additional image information in the form of for example a hemoglobin content of the observation region. For example, such a hemoglobin content is superimposed on the other pieces of image information as a color image pixel-by-pixel here. In this case, the display unit may also comprise the evaluation unit therein, with the result that there is no need for separate equipment for the display unit and the evaluation unit.

The invention is explained in more detail below using exemplary embodiments. In the drawing:

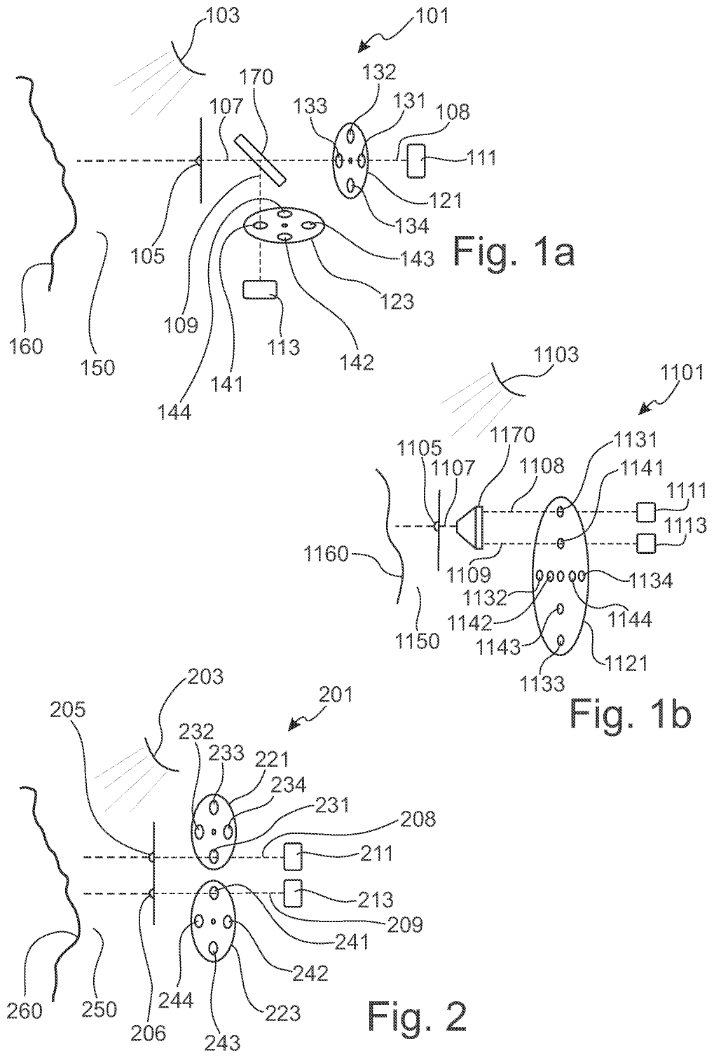

FIG. 1a shows a schematic illustration of an endoscope system having a beam splitter for two optical paths in a side view,

FIG. 1b shows a schematic illustration of an endoscope system having a beam splitter for two optical paths and a joint filter wheel in a side view,

FIG. 2 shows a schematic illustration of an endoscope system with two separate optical paths in a side view,

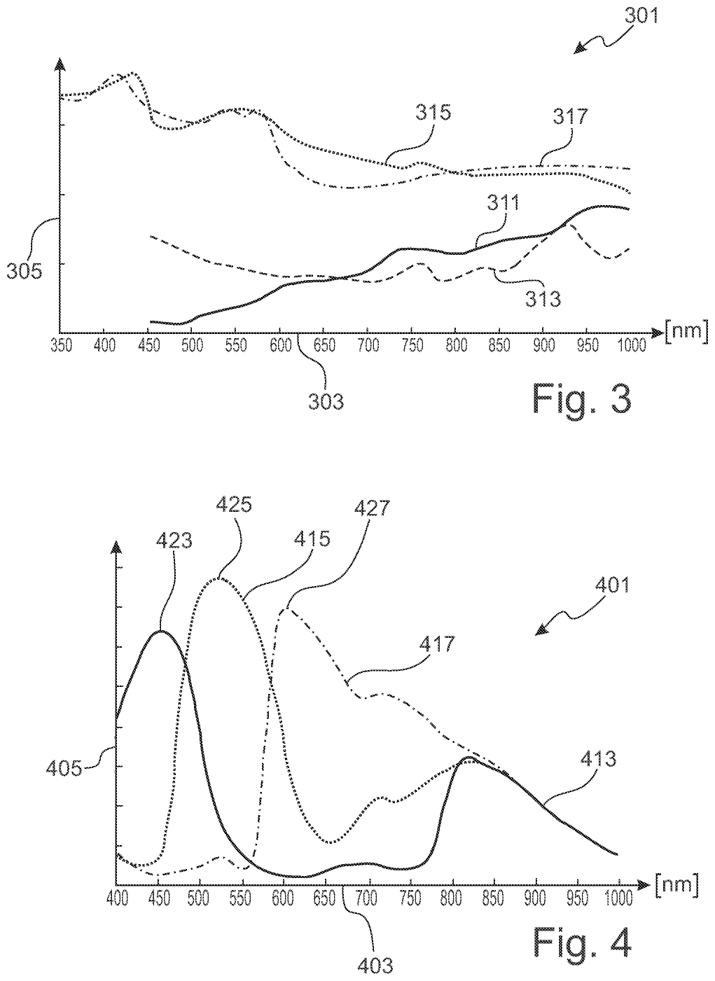

FIG. 3 shows a diagram regarding the spectral absorption of light by various molecules for determining physiological parameters of the tissue,

FIG. 4 shows a diagram regarding the color-dependent sensitivity of an exemplary image sensor having a Bayer filter,

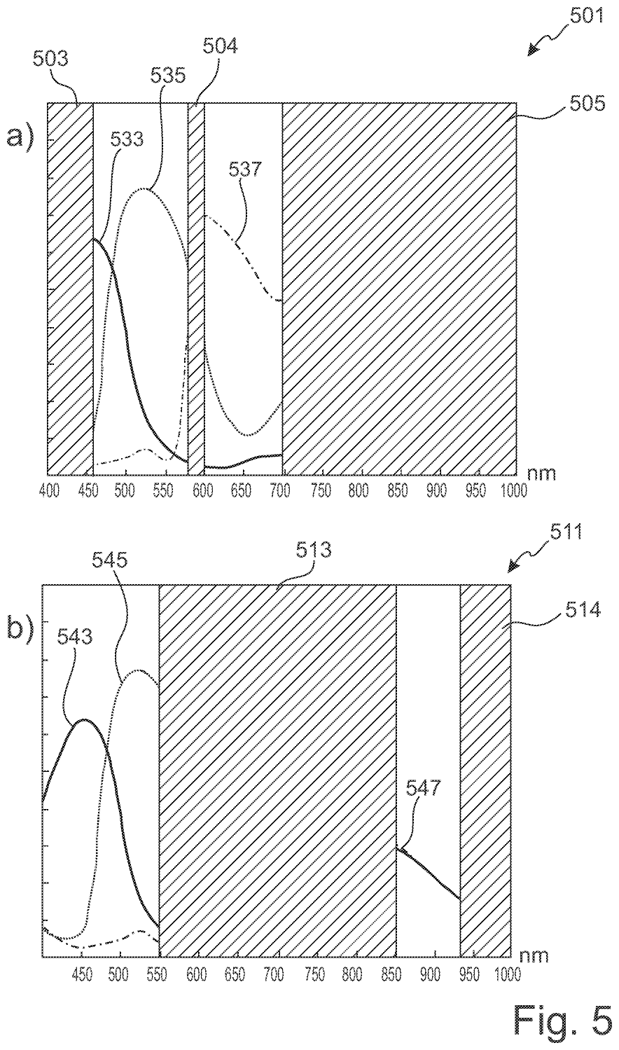

FIGS. 5a/5b each show a diagram for a first image sensor and a second image sensor of the endoscope systems of FIGS. 1a/1b or FIG. 2 with a filter spectrum of a first mode of operation applied thereto in each case,

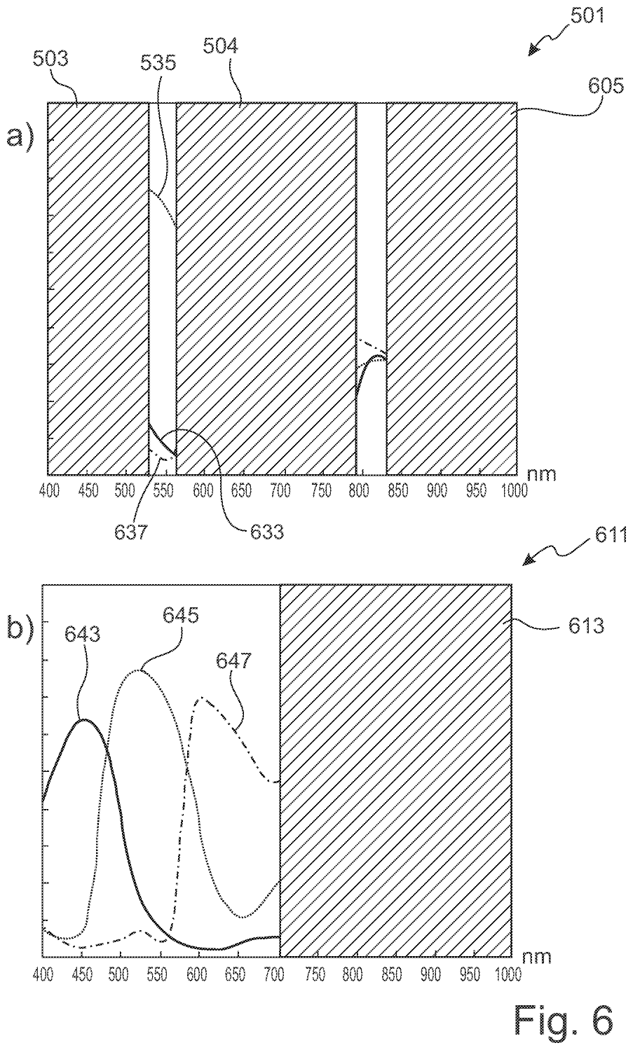

FIGS. 6a/6b each show a diagram for a first image sensor and a second image sensor of the endoscope systems of FIGS. 1a/1b or FIG. 2 with a filter spectrum of a second mode of operation applied thereto in each case,

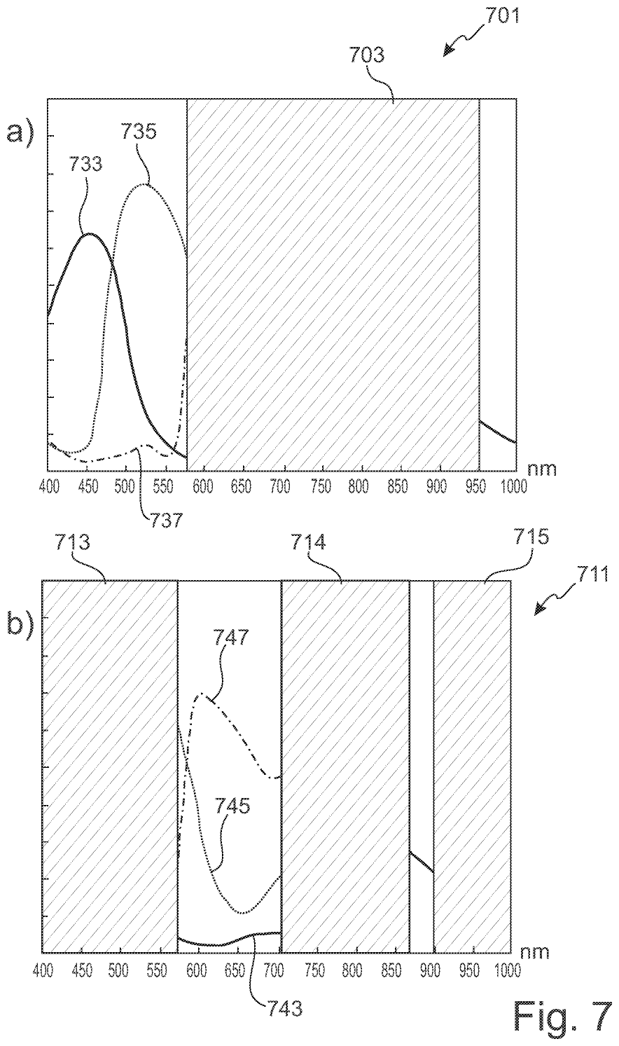

FIGS. 7a/7b each show a diagram for a first image sensor and a second image sensor of the endoscope systems of FIGS. 1a/1b or FIG. 2 with a filter spectrum of a third mode of operation applied thereto in each case,

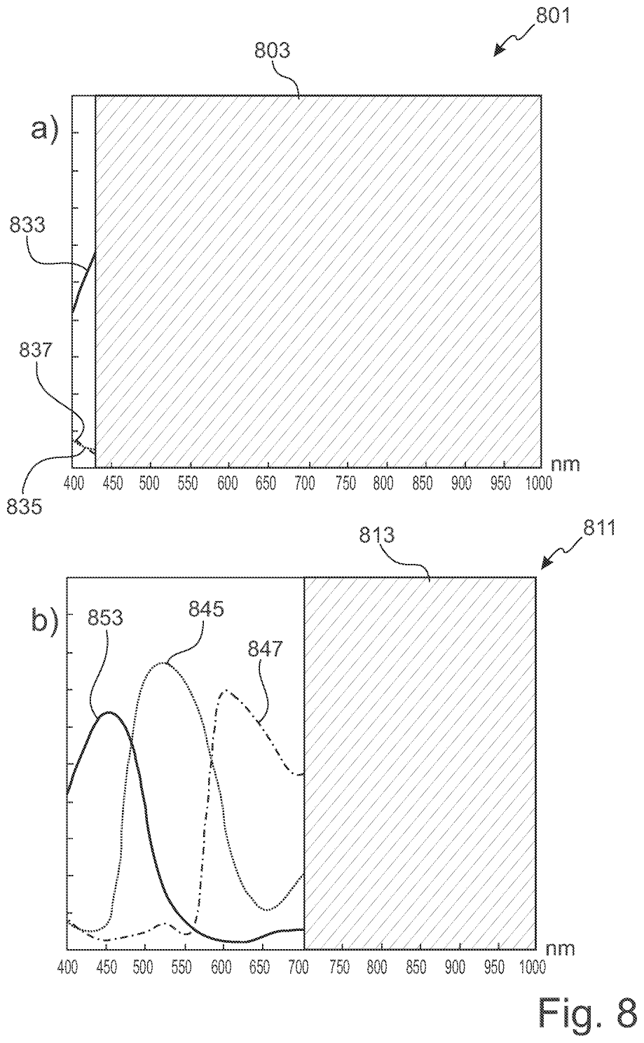

FIGS. 8a/8b each show a diagram for a first image sensor and a second image sensor of the endoscope systems of FIGS. 1a/1b or FIG. 2 with a filter spectrum of a fourth mode of operation applied thereto in each case, and

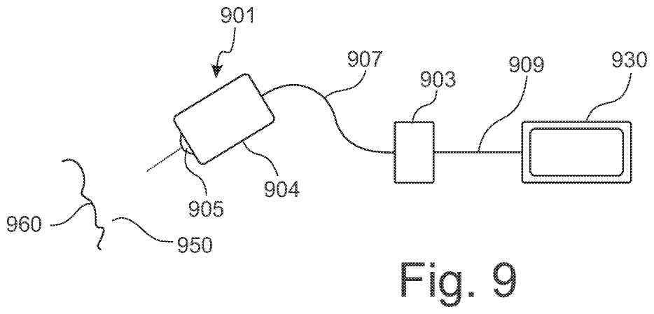

FIG. 9 shows an arrangement of an exemplary endoscope having an endoscope system of FIGS. 1a/1b or FIG. 2, a computer and an electronic visual display for visualizing an image of an observation region.

An endoscope system 101 serves to pictorially capture an object 160 in an observation region 150. In this case, the endoscope system 101 represents, by way of example, a corresponding image recording system of a medical imaging apparatus, for example of an endoscope or an exoscope. The endoscope system 101 comprises a light source 103 for illuminating the observation region 150 and the object 160 with white light. A lens 105 in an optical path 107 receives incident light from the observation region 150 and transmits said light to a semi-transparent mirror 170 arranged at the angle of 45° along the optical path 107. Light incident along the optical path 107 is guided along an optical path 108 to an image sensor 111 and along an optical path 109 to an image sensor 113 in equal parts by way of the semi-transparent mirror 170. The image sensor 111 and the image sensor 113 are each designed as RGB sensors with a Bayer filter and high sensitivity in the near infrared range.

A filter wheel 121 is arranged in the optical path 108. The filter wheel 121 is rotatably arranged along an axis parallel to the optical path 108 and in such a way that a filter 131, a filter 132, a filter 133, or a filter 134 are selectively introducible into the optical path 108. To this end, the filter 131, the filter 132, the filter 133, and the filter 134 are arranged on the filter wheel 121 along the same radii. Twisting the filter wheel 121 thus allows a respective filter to be introduced into the optical path 108, with the result that light incident from the observation region 150 along the optical path 108 reaches the image sensor 111 in filtered fashion.

Analogously, a filter wheel 123 carrying a filter 141, a filter 142, a filter 143, and a filter 144 is arranged in the optical path 109. In a manner analogous to the filter wheel 121, the filter wheel 123 can thus be used to introduce a respective filter into the optical path 109 by means of a rotation about an axis of rotation running parallel to the optical path 109, with the result that light originating from the observation region 150 is incident on the image sensor 113 in filtered fashion.

The filter 131, the filter 132, the filter 133, and the filter 134 of the filter wheel 121 and also the filter 141, the filter 142, the filter 143, and the filter 144 of the filter wheel 123 have respective different filter properties; accordingly, a respective filter filters out different wavelength ranges of the light incident along the respective optical path from the observation region 150 and allows remaining constituents, which is to say remaining wavelength ranges of the incident light, to pass.

An alternative setup of an endoscope system 1101 likewise serves to pictorially capture an object 1160 in an observation region 1150. In this case, the endoscope system 1101 represents, by way of example, a corresponding image recording system of a medical imaging apparatus, for example of an endoscope or an exoscope. The endoscope system 1101 comprises a light source 1103 for illuminating the observation region 1150 and the object 1160 with white light. A lens 1105 in an optical path 1107 receives incident light from the observation region 1150 and transmits said light to a beam splitter 1170. Light incident along the optical path 1107 is guided along an optical path 1108 to an image sensor 1111 and along an optical path 1109 to an image sensor 1113 in equal parts by way of the beam splitter 1170. The image sensor 1111 and the image sensor 1113 are each designed as RGB sensors with a Bayer filter and high sensitivity in the near infrared range.

In the present case, the beam splitter 1170 shown is implemented by means of a plurality of prisms. In an alternative, a different arrangement for splitting the incident light can also be used here, for example an arrangement consisting of a plurality of mirrors, wherein an expedient mechanical arrangement of the components, for example the filter wheel 1121 in relation to the optical path 1107, can be chosen in each case.

A joint filter wheel 1121 is arranged along the optical paths 1108 and 1109. The filter wheel 1121 is rotatably arranged along an axis parallel to the optical path 1108 and optical path 1109 and in such a way that a filter 1131 together with a filter 1141, a filter 1132 together with a filter 1142, a filter 1133 together with a filter 1143, or a filter 1134 together with a filter 1144 are selectively introducible into the optical path 1108 and into the optical path 1109. To this end, the filter 1131, the filter 1132, the filter 1133, and the filter 1134 are arranged on the filter wheel 1121 along a radius in a position corresponding to the optical path 1108, and the filter 1141, the filter 1142, the filter 1143, and the filter 1144 are arranged on said filter wheel along another radius in a position corresponding to the optical path 1109. Twisting the filter wheel 121 thus allows a respective pair of filters to be jointly introduced into the optical path 1108 and the optical path 1109, with the result that light from the observation region 1150 reaches the image sensor 1111 in filtered fashion along the optical path 1108 and reaches the image sensor 1113 in filtered fashion along the optical path 1109.

The filter 1131, the filter 1132, the filter 1133, and the filter 1134 and also the filter 1141, the filter 1142, the filter 1143, and the filter 1144 of the filter wheel 1121 have respective different filter properties; accordingly, a respective filter filters out different wavelength ranges of the light incident along the respective optical path from the observation region 1150 and allows remaining constituents, which is to say remaining wavelength ranges of the incident light, to pass. In this context, it should be observed that, both above and in the application example following below, the description according to the wording provides for a sharp separation between a transmission and a retention of corresponding wavelengths at corresponding wavelengths; it is self-evident here that blurring and corresponding transitions arising on account of the technology need to be considered but are not explicitly mentioned below for reasons of clarity.

In this case, the illustration in FIGS. 1a, 1b, and 2 with regards to respective filter wheels already mentioned above or specified below was chosen such that a functionally comprehensible illustration was made possible while accepting perspective inaccuracies, and so, in particular, the respective filter wheels are shown in perspective with a slight distortion in an illustration otherwise chosen in the form of a schematic side view. Respective optical paths always run through a filter respectively in engagement or through a plurality of filters respectively in engagement, with the result that incident light is incident on a respective image sensor in filtered fashion in each case.

In a manner analogous to the endoscope system 101, an endoscope system 201 serves to observe an object 260 in an observation region 250. However, in contrast to the endoscope system 101 or to the endoscope system 1101, no semi-transparent mirror is used as a beam splitter or no other beam splitter in this exemplary embodiment, but instead optical paths arranged separately from one another.

The endoscope system 201 comprises a light source 203, by means of which the observation region 250 and hence the object 260 are illuminated with white light. Light from the object 260 from the observation region 250 travels along a respective optical path 208 and 209 into the endoscope system 201 through a lens 205 and a lens 206, which are arranged next to one another. In this case, light incident through the lens 205 propagates along an optical path 208 to an image sensor 211, while light incident through the lens 206 propagates along an optical path 209 to an image sensor 213. The image sensor 211 and the image sensor 213 are each designed as RGB sensors with a Bayer filter and high sensitivity in the near infrared range.

A filter wheel 221 carrying a filter 231, a filter 232, a filter 233, and a filter 234, is arranged in the optical path 208 between the lens 205 and the image sensor 211. In this case, the respective filters are arranged at the same radius around an axis of rotation of the filter wheel 221, with the result that a respective filter is introducible into the optical path 208 by means of a rotation of the filter wheel 221. Consequently, light coming from the observation region 250 and filtered by means of the respective filter is then incident on the image sensor 211. In a manner analogous to the previous example, the axis of rotation of the filter wheel 221 is in this case arranged parallel to the optical path 203.

Light from the observation region 250 traveling through the lens 206 in the direction of the image sensor 213 is guided analogously through a filter wheel 223. The filter wheel 223 carries respective filters 241, 242, 243, and 244 which are arranged along the same radii in a manner analogous to the filter wheel 221, and so a rotation of the filter wheel 223 allows a respective filter to be introduced into the optical path 209. The light incident along the optical path 209 is then filtered by a respective filter located in the optical path 209 and only wavelength ranges transmitted by the filter are incident on the image sensor 213.

In a respective alternative, the filters, for example the filters 231, 232, 233, and 234, can in this case also be introduced jointly or separately from one another into the respective optical path by means of pivoting in or tilting in. This applies analogously to all filter wheels described above. In the following, the assumption is made that the respective filter wheels 121, 123, 221, and 223 are controlled in such a way that filter pairs, assigned respectively to one another, on different filter wheels are twisted into the respective optical paths jointly with one another, i.e., for example, a filter 131 and a filter 141 as a first group, a filter 132 and a filter 142 as a second group, and so on in analogous fashion for further filters, are jointly controllable in a respective group and jointly introducible into the respective optical paths. Different combinations of the respective filters are also possible in this context; the present list only serves as an example here.

A diagram 301 has an abscissa axis 303 and an ordinate axis 305. In this case, the abscissa axis 303 describes a wavelength of light, formed between 350 nm and 1000 nm in the diagram 301. In this case, the associated ordinate axis 305 describes an absorption coefficient, associated with a wavelength of light and depicted qualitatively, of a certain molecule. In this case, the ordinate axis has a logarithmic scale. For example, the ordinate axis 305 could in this case map a region between 0 and 1, but it may also have a different scaling. Reference is made to qualitative features for the present example.

The diagram 301 shows a wavelength dependence of the absorption coefficient of a molecule 311, a wavelength dependence of the absorption coefficient of a molecule 313, a wavelength dependence of the absorption coefficient of a molecule 315, and a wavelength dependence of the absorption coefficient of a molecule 317. The respective wavelength dependencies of the absorption coefficients are usable to draw conclusions about the composition of human tissue for light of appropriate wavelengths reflected by said tissue, for example. Thus, the wavelength dependence of the absorption coefficient 311 can be used to determine the water content in the tissue, the wavelength dependence of the absorption coefficient 313 can be used to determine a fat content in the tissue, the wavelength dependence of the absorption coefficient 315 can be used to determine the proportion of deoxygenated hemoglobin in the tissue, and the wavelength dependence of the absorption coefficient 317 can be used to determine the proportion of oxygenated hemoglobin in the tissue.

Consequently, in the case of an appropriate irradiation of the tissue with light at specific wavelengths, a corresponding reaction of the tissue within the meaning of a reflection in accordance with the wavelength dependencies, labeled above, of absorption coefficients of specific molecules 311, 313, 315, and 317 can be expected, from which in turn it is then possible to deduce the properties of the tissue.

A diagram 401 has an abscissa axis 403 and an ordinate axis 405. In this case, the abscissa axis 403 describes a respective wavelength of light between 400 nm and 1000 nm. The ordinate axis 405 describes a corresponding light sensitivity qualitatively, which is to say for example between 0 and 1. Hence, the diagram 401 shows the light sensitivity of an RGB sensor with an appropriate sensor filter used for this example. Thus, the image sensor 111, the image sensor 113, the image sensor 211, and the image sensor 213 have a corresponding sensitivity distribution in accordance with the diagram 401. Other RGB sensors with other sensor filters can have sensitivity distributions that deviate therefrom.

To this end, the diagram 401 shows a function 413 which describes the sensitivity of a respective image sensor to blue light, a function 415 which describes a sensitivity of the respective RGB sensor to green light, and a function 417 which describes a sensitivity of the respective RGB sensor to red light. The sensitivity of the RGB sensor which forms the basis of this example to blue light has a maximum 423 at approximately 450 nm, the sensitivity to green light has a maximum 425 at approximately 520 nm, and the sensitivity to red light has a maximum 427 at approximately 600 nm. In this context, other RGB sensors may have different properties.

By means of the above-described respective groups of different filters in the different mechanical arrangements it is now possible to switch into and/or set different modes of operation for the respective endoscope system. Here, the following description relates to the endoscope systems 101, 1101, and 201 since the light incident on the respective image sensors is decisive for corresponding effects, independently of whether corresponding light reaches the respective image sensor by means of a beam splitter, by means of different filter arrangements, or on completely different optical paths. Therefore, what is described below in relation to the image sensor 111 also applies to the image sensor 1111 and the image sensor 211, and what is described below in relation to the image sensor 113 also applies analogously to the image sensor 1113 and the image sensor 213.

Under these conditions, four different modes of operation are described below for clarification with reference being made by way of example to the endoscope system 101:

For example, the filters 131 and 141 are introduced into the optical paths 108 and 109, respectively, for a first mode of operation. In this case, a diagram 501 shows the light spectrum incident on the image sensor 111 along the optical path 108 as a result of the filter 131, while a diagram 511 shows the light spectrum incident on the image sensor 113 as a result of the filter 141. Consequently, differently filtered light spectra are incident on the image sensor 111 and the image sensor 113. In this context, the diagram 501 shows a blocked spectral range 503, a blocked spectral range 504, and a blocked spectral range 505. The blocked spectral range 503 prevents light below 460 nm from reaching the image sensor 111. The blocked spectral range 504 prevents light between 580 nm and 600 nm from reaching the image sensor 111. The blocked spectral range 505 prevents light at a wavelength above 700 nm from reaching the image sensor 111. Consequently, only light at a wavelength between 460 nm and 580 nm and a wavelength between 600 nm and 700 nm reaches the image sensor 111. In this case, the respective curves 533, 535, and 537 correspond to the curves 413, 415, and 417 for the different sensitivities according to colors of the RGB image sensor 111.

Analogously, the diagram 511 shows a blocked spectral range 513 and a blocked spectral range 514. The blocked spectral range 513 prevents light between 550 nm and 850 nm from reaching the image sensor 113; the blocked spectral range 514 prevents light at a wavelength above 930 nm from reaching the image sensor 113. Therefore, only light at a wavelength up to 550 nm and between 850 nm and 930 nm reaches the image sensor 113. The functions 543, 545, and 547 correspond to the functions 413, 415, and 417 for the different sensitivities according to colors of the RGB image sensor 113.

The red information from the image sensor 111, the green information from the image sensor 111, and a difference made of the blue information from the image sensor 113 minus the red information from the image sensor 113 can be used to calculate an RGB image from the pieces of image information filtered thus, and this RGB image can be displayed to an operator or user of the respective endoscope system such that this operator or user can see a real image of the object 160 or, in a manner analogous thereto in the other arrangements according to the invention, of the object 1160 or for example of the object 260.

Furthermore, an oxygenation of the tissue of the object 160, 1160, or 260 can be read from different wavelength ranges. Thus, an oxygenation can be read in the visible range from the blue information from the image sensor 111 from the transmitted light spectrum between 460 nm and 580 nm; this process can also be implemented using the red information from the image sensor 111 in the wavelength range between 600 nm and 700 nm.

In parallel or complementary or else alternative fashion, an oxygenation of the tissue can be read in the near infrared range from the red information from the image sensor 111 between 600 nm and 700 nm. Likewise, the red information from the image sensor 113 in the wavelength range between 850 nm and 930 nm can be used to this end. Consequently, both an RGB image and the oxygenation of the tissue can be read out in this first mode. To this end, the ratio of the absorption coefficients of oxygenated and deoxygenated hemoglobin, which changes depending on the wavelength, is used by virtue of the oxygenation being deduced from the ratio at 600 nm to 700 nm and the inverse ratio at approximately 850 nm to 930 nm.

For example, the filters 132 and 142 are introduced into the optical paths 108 and 109, respectively, for a second mode of operation. In this case, a diagram 601 shows the light spectrum obtained along the optical path 108 as a result of the filter 132, while a diagram 611 shows the light spectrum obtained as a result of the filter 142. Consequently, differently filtered light spectra are incident on the image sensor 111 and the image sensor 113. In this context, the diagram 601 shows a blocked spectral range 603, a blocked spectral range 604, and a blocked spectral range 605. The blocked spectral range 603 prevents light below 530 nm from reaching the image sensor 111. The blocked spectral range 604 prevents light between 560 nm and 785 nm from reaching the image sensor 111. The blocked spectral range 605 prevents light at a wavelength above 825 nm from reaching the image sensor 111. Consequently, only light at a wavelength between 530 nm and 560 nm and a wavelength between 785 nm and 825 nm reaches the image sensor 111. The functions 633, 635, and 637 correspond to the functions 413, 415, and 417 for the different sensitivities according to colors of the RGB image sensor 111.

In a manner analogous thereto, the diagram 611 shows a blocked spectral range 613. The blocked spectral range 613 prevents light above 700 nm from reaching the image sensor 113. Therefore, only light at a wavelength up to 700 nm reaches the image sensor 113. The functions 643, 645, and 647 correspond to the functions 413, 415, and 417 for the different sensitivities according to colors of the RGB image sensor 113.

The red information from the image sensor 113, the green information from the image sensor 113, and the blue information from the image sensor 113 can be used to calculate an RGB image from the pieces of image information filtered thus, and this RGB image can be displayed to an operator or user of the respective endoscope system such that this operator or user can see a real image of the object 160, 1160 or for example of the object 260. In this respect, it is evident from the diagram 611 that all maxima analogous to the maxima 423, 425, and 427 of the sensor sensitivities are transmitted by the filter and hence evaluable.

Furthermore, a hemoglobin index of the tissue of the object 160 can be read from different wavelength ranges. Thus, the hemoglobin content can be read from the green information from the image sensor 111 minus the blue information from the sensor 111 from the transmitted light spectrum between 530 nm and 560 nm; this process can also be implemented using the red information from the image sensor 111 in the wavelength range between 785 nm and 825 nm. In this respect, for example the equal absorption (isosbestic points) by oxygenated and deoxygenated hemoglobin at approx. 540 nm and approx. 800 nm (cf. wavelength dependencies 315 and 317) can be used to derive a quotient for the statement regarding the hemoglobin content.

For example, the filters 133 and 143 are introduced into the optical paths 108 and 109, respectively, for a third mode of operation. In this case, a diagram 701 shows the light spectrum attained along the optical path 108 as a result of the filter 133, while a diagram 711 shows the light spectrum attained as a result of the filter 143. Consequently, differently filtered light spectra are incident on the image sensor 111 and the image sensor 113. In this case, the diagram 701 shows a blocked spectral range 703. The blocked spectral range 703 prevents light between 575 nm and 950 nm from reaching the image sensor 111. Consequently, only light at a wavelength up to 575 nm and a wavelength above 950 nm reaches the image sensor 111. The curves 733, 735, and 737 correspond to the curves 413, 415, and 417 for the different sensitivities according to colors of the RGB image sensor 111.

Analogously, the diagram 711 shows a blocked spectral range 713, a blocked spectral range 714, and a blocked spectral range 715. The blocked spectral range 713 prevents light below 575 nm from reaching the image sensor 113, the blocked spectral range 714 prevents light at a wavelength between 700 nm and 875 nm from reaching the image sensor 113, and the blocked spectral range 715 prevents light at a wavelength above 895 nm from reaching the image sensor 113. Therefore, only light at a wavelength between 575 nm and 700 nm and between 875 nm and 895 nm reaches the image sensor 113.

A difference made up of the red information from the image sensor 113 minus the blue information from the image sensor 113 serving to obtain a calculated piece of red information, a difference made up of the green information from the image sensor 111 minus the red information from the image sensor 111 serving to obtain a calculated piece of green information, and a difference made up of the blue information from the image sensor 111 minus the red information from the image sensor 111 serving to obtain a calculated piece of blue information can be used to calculate an RGB image from the pieces of image information filtered thus, and this RGB image can be displayed to an operator or user of the respective endoscope system such that this operator or user can see a real image of the object 160, 1160 or for example of the object 260.

Furthermore, a water content of the tissue of the object 160 can be read from different wavelength ranges. Thus, the blue information of the image sensor 113 can be used to read a water content from the transmitted light spectrum between 875 nm and 895 nm, wherein transmitted light of the light spectrum of the measured intensity between 575 nm and 700 nm, which likewise is incident on the image sensor 113, is negligible on account of a very low sensitivity of the blue channel of the image sensor 113 in this wavelength range; this process can likewise be implemented using the red information from the image sensor 111 in the wavelength range between 950 nm and 1000 nm, wherein incident light in the wavelength range between 400 nm and 575 nm is likewise negligible. Thus, a wavelength range between 875 nm and 895 nm is recorded in order to use the approximately uniform profile with regards to the absorbance of water for reference purposes. A readout of the wavelength range between 950 nm and 1000 nm, in which an increasing absorbance of water is present, serves as a basis for establishing the water content in the tissue.

For example, the filters 134 and 144 are introduced into the optical paths 108 and 109, respectively, for a fourth mode of operation. In this case, a diagram 801 shows the light spectrum attained along the optical path 108 as a result of the filter 134, while a diagram 811 shows the light spectrum attained as a result of the filter 144. Consequently, differently filtered light spectra are incident on the image sensor 111 and the image sensor 113. In this case, the diagram 801 shows a blocked spectral range 803. The blocked spectral range 803 prevents light above 430 nm from reaching the image sensor 111. Consequently, only light at a wavelength up to 430 nm reaches the image sensor 111.

In a manner analogous thereto, the diagram 811 has a blocked spectral range 813. The blocked spectral range 813 prevents light above 700 nm from reaching the image sensor 113. Therefore, only light at a wavelength up to 700 nm reaches the image sensor 113.

The red information from the image sensor 113, the green information from the image sensor 113, and the blue information from the image sensor 113 can be used to read out an RGB image from the pieces of image information filtered thus, and this RGB image can be displayed to an operator or user of the respective endoscope system such that this operator or user can see a real image of the object 160, 1160 or for example of the object 260. The respective maxima of the sensitivities of the image sensor 113 analogous to the maxima 423, 425, and 427 in the diagram 401 are readable here in unfiltered fashion.

Further, an increase in contrast of blood vessels within a tissue in an image of the object 160 can be read from the filtered pieces of image information by means of what is known as “narrow-band imaging” (NBI). For this purpose, contrast can be increased by reading the blue information from the image sensor 111 in the range between 400 nm and 430 nm. This is facilitated by the high absorption of light by the oxygenated hemoglobin and the deoxygenated hemoglobin, whereby blood vessels in the image appear darker than surrounding tissue.

Consequently, the presented respective arrangement can, by means of the endoscope system 101, 1101 or else the endoscope system 201, implement a switchover between the respective modes for an introduction of different filters or different groups of filters, in order to display, in parallel, both a real RGB image of a respective observation region and a piece of additional information created in accordance with the respective mode, for example regarding a hemoglobin content or an oxygen content in the observation region.

Additionally, a stereoscopic image can also be reconstructed from the image from the image sensor 111 and the image from the image sensor 113, with the result that there is a three-dimensional image of the object 160, 1160, and/or 260 in the observation region 150, 1150, and/or 250.

An endoscope camera 901 comprises a housing 904 and a lens 905. The endoscope system 101 is accommodated within the housing; in this example, the lens 905 corresponds to the lens 105 here. The image data read out from the respective image sensor are transmitted to a computer 903 by means of a cable 907. The computer 903 evaluates the corresponding image data and displays these on an electronic visual display 930 for a user (not depicted) via a cable 909.

### LIST OF REFERENCE SIGNS

101 Endoscope system
103 Light source
105 Lens
107 Optical path
108 Optical path
109 Optical path
111 Image sensor
113 Image sensor
121 Filter wheel
123 Filter wheel
131 Filter
132 Filter
133 Filter
134 Filter
141 Filter
142 Filter
143 Filter
144 Filter
150 Observation region
160 Object
170 Semi-transparent mirror
201 Endoscope system
203 Light source
205 Lens
206 Lens
208 Optical path
209 Optical path
211 Image sensor
213 Image sensor
221 Filter wheel
223 Filter wheel
231 Filter
232 Filter
233 Filter
234 Filter
241 Filter
242 Filter
243 Filter
244 Filter
250 Observation region
260 Object
301 Diagram
303 Abscissa axis
305 Ordinate axis
311 Wavelength dependence
313 Wavelength dependence
315 Wavelength dependence
317 Wavelength dependence
401 Diagram
403 Abscissa axis
405 Ordinate axis
413 Function
415 Function
417 Function
423 Maximum
425 Maximum
427 Maximum
501 Diagram
503 Blocked spectral range
504 Blocked spectral range
505 Blocked spectral range
511 Diagram
513 Blocked spectral range
514 Blocked spectral range
533 Function
535 Function
537 Function
543 Function
545 Function
547 Function
601 Diagram
603 Blocked spectral range
604 Blocked spectral range
605 Blocked spectral range
611 Diagram
613 Blocked spectral range
633 Function
635 Function
637 Function
643 Function
645 Function
647 Function
701 Diagram
703 Blocked spectral range
711 Diagram
713 Blocked spectral range
714 Blocked spectral range
715 Blocked spectral range
733 Function
735 Function
737 Function
743 Function
745 Function
747 Function
801 Diagram
803 Blocked spectral range
811 Diagram
813 Blocked spectral range
833 Function
835 Function
837 Function
843 Function
845 Function
847 Function
901 Endoscope camera
903 Computer
904 Housing
905 Lens
907 Cable
909 Cable
930 Electronic visual display
950 Observation region
960 Object
1101 Endoscope system
1103 Light source
1105 Lens
1107 Optical path
1108 Optical path
1109 Optical path
1111 Image sensor
1113 Image sensor
1121 Filter wheel
1131 Filter
1132 Filter
1133 Filter
1134 Filter
1141 Filter
1142 Filter
1143 Filter
1144 Filter
1150 Observation region
1160 Object

1. A medical imaging apparatus, in particular an endoscope or an exoscope, having a light source with a light spectrum, an optical unit with a first optical path and a first image sensor with a first sensor filter, and a second optical path with a second image sensor with a second sensor filter, wherein the respective optical path extends between an observation region and the respective image sensor, such that the first image sensor records a first image of the observation region by means of the first optical path and the second image sensor records a second image of the observation region by means of the second optical path, wherein the light source is configured to illuminate the observation region with the light spectrum and the first optical path is assigned a first filter with a first filter spectrum and/or the second optical path is assigned a second filter with a second filter spectrum such that physiological parameters of the observation region are determinable depending on the light spectrum by means of a respective filtered piece of image information from the first image and/or the second image, wherein that the first filter is introducible into the first optical path by means of a first filter positioning device and/or the second filter is introducible into the second optical path by means of a second filter positioning device such that a filtered piece of image information from the first image and/or a filtered piece of image information from the second image, depending on the respective filter introduced into the respective optical path, render/renders evaluable different spectral regions of the respective image in order to obtain one piece of additional image information or more pieces of additional image information regarding the physiological parameters in the observation region.

2. The medical imaging apparatus as claimed in claim 1, wherein the first optical path and/or the second optical path is or are assigned a third filter a fourth filter, and/or a further filter, wherein the third filter is introducible by means of a third filter positioning device, the fourth filter is introducible by means of a fourth filter positioning device, and/or the further filter is introducible by means of a further filter positioning device W, into the first optical path and/or into the second optical path.

3. The medical imaging apparatus as claimed in claim 1, wherein the first filter, the second filter, the third filter, the fourth filter, and/or the further filter have different filter spectra from one another.

4. The medical imaging apparatus as claimed in claim 1, wherein the first filter, the second filter, the third filter, the fourth filter, and/or the further filter are grouped in a group, in particular in a group of two filters assigned to one another, such that filters in each case assigned to one another in the group are jointly introducible into the first optical path and into the second optical path.

5. The medical imaging apparatus as claimed in claim 1, wherein the first filter, the second filter, the third filter, the fourth filter, and/or the further filter are grouped with one another in an operational group, in particular in a respective operational group made of two filters assigned to one another, such that a first mode of operation, a second mode of operation, a third mode of operation, and/or a further mode of operation is selectable by a user by means of an input, wherein a selection can be made from a group of respective filters for different modes of operation, for example a group of the first filter and the second filter for an oxygen saturation mode of operation, a group of the third filter and the fourth filter for a water content mode of operation, and/or a group of two further filters for a hemoglobin content mode of operation.

6. The medical imaging apparatus as claimed in claim 4, wherein the filter positioning device associated with a respective mutually assigned filter is assigned to a filter positioning system and operatively connected to the latter such that the respective group or operational group of in particular two filters assigned to one another is jointly introducible into the first optical path and into the second optical path by means of the filter positioning system.

7. The medical imaging apparatus as claimed in claim 4, wherein continuous imaging is implemented in such a way that a group and/or an operational group is selected and there is an evaluation with regards to the physiological parameters together with the recording and/or in parallel with the recording of the image information, in particular in real time.

8. The medical imaging apparatus as claimed in claim 1, wherein the respective filter or the respective filters is or are introducible into the respective optical path by means of being pushed in, by means of being pivoted in, and/or by means of being screwed in.

9. The medical imaging apparatus as claimed in claim 1, wherein the respective filter positioning device or the respective filter positioning system comprises or comprise a drive such that the respective filter or the filters assigned respectively to one another is or are introducible into the first optical path and/or into the second optical path by means of the respective drive, wherein the drive in particular comprises a stepper motor, a gearmotor, a lifting magnet, a pivoting magnet, and/or a manually operable drive.

10. The medical imaging apparatus as claimed in claim 1, wherein the first filter, the second filter, the third filter, the fourth filter, and/or the further filter is an edge filter or a band filter.

11. The medical imaging apparatus as claimed in claim 1, wherein the first filter, the second filter, the third filter, the fourth filter, and/or the further filter have a respective filter spectrum such that by means of the different filter spectra of the respective filters by means of two filters assigned respectively to one another in the first optical path and in the second optical path it is possible, by way of a parallel evaluation of the first image and the second image, to determine a color image of the observation region and to determine one piece of additional image information or more pieces of additional image information regarding the physiological parameters together.

12. The medical imaging apparatus as claimed in claim 1, wherein the light source is a white-light source, and so the light source illuminates the observation region with white light.

13. The medical imaging apparatus as claimed in claim 1, wherein the optical unit comprises a beam splitter, wherein the beam splitter, is used to split an image of the observation region into the first image and the second image such that the first image is suppliable to the first image sensor via the first optical path and the second image is suppliable to the second image sensor via the second optical path, wherein the beam splitter in particular comprises a prism, a semi-transparent mirror or two semi-transparent mirrors, and/or a mirror.

14. The medical imaging apparatus as claimed in claim 1 wherein the first optical path and the second optical path are arranged spatially offset from one another such that the first image sensor records a first image of the observation region by means of the first optical path and the second image sensor records a second image of the observation region by means of the second optical path.

15. The medical imaging apparatus as claimed in claim 14, wherein the first image and the second image are assigned to one another in an overlaid image to form a piece of dual image information, wherein in particular the formation of the piece of dual image information in the overlaid image comprises a reconstruction of a correlation between the first image and the second image on the basis of a piece of respective filtered image information, in particular a reconstruction of a disparity, wherein the reconstruction is implemented in particular on the basis of pieces of image information with wavelength spectra transmitted by the respective filter, in particular as a result of illuminating the observation region with white light.

16. The medical imaging apparatus as claimed in claim 15, wherein the formation of the piece of dual image information comprises a stereoscopic formation of a piece of spatial image information for the observation region.

17. The medical imaging apparatus as claimed in claim 1, wherein an evaluation unit which is configured to evaluate the first image and/or the second image in relation to a hemoglobin index, a water index, an oxygen index or an oxygen-infrared index, with the result that a hemoglobin content, a water content, an oxygen concentration, and/or a presence of an adjuvant, in particular a fluorescent substance, is determinable.

18. The medical imaging apparatus as claimed in claim 1 wherein a display unit which is configured for continuous, parallel, simultaneous, overlaid and/or correlated display of the piece of first image information, the piece of second image information, the piece of dual image information, the stereoscopically formed piece of spatial image information, and/or the piece of additional image information.
